(19) United States
(12) Patent Application Publication
(10) Pub. No.: US 2024/0335650 A1
(43) Pub. Date: Oct. 10, 2024

(54) CIRCULATORY SUPPORT DEVICE SYSTEM

(71) Applicant: Boston Scientific Scimed, Inc., Maple Grove, MN (US)

(72) Inventors: Judith Tiferes Wang, Kenton, OH (US); Spencer Fodness-Bondhus, Columbia Heights, MN (US); Corydon Carlson, Stillwater, MN (US)

(73) Assignee: Boston Scientific Scimed, Inc., Maple Grove, MN (US)

(21) Appl. No.: 18/627,919

(22) Filed: Apr. 5, 2024

Related U.S. Application Data
(60) Provisional application No. 63/457,935, filed on Apr. 7, 2023.

(51) Int. Cl. A61M 60/585; A61M 60/216; A61M 60/414

(52) U.S. Cl. CPC A61M 60/585; A61M 60/216; A61M 60/414; A61M 2205/04; A61M 2205/10; A61M 2205/3303; A61M 2205/3327; A61M 2205/3331; A61M 2205/3365; A61M 2205/505; A61M 2210/125; A61M 2230/30

(57) ABSTRACT

Example cardiac pump systems are disclosed. An example cardiac pump system includes a cardiac pump coupled to a console. Further, the console includes a first display including a first visual representation of a first parameter of the cardiac pump system. Further, the first display includes a first status indicator and a second status indicator corresponding to the first parameter. Further, the first display is configured to display the first status indicator when the first parameter satisfies a first condition, and wherein the first display is configured to display the second status indicator when the first parameter satisfies a second condition.

### CROSS-REFERENCE TO RELATED APPLICATIONS

This application claims the benefit of priority under 35 U.S.C. § 119 of U.S. Provisional Application No. 63/457,935, filed Apr. 7, 2023, the entire disclosure of which is hereby incorporated by reference.

### TECHNICAL FIELD

The present disclosure relates to percutaneous circulatory support device systems. More specifically, the disclosure relates to a visual display of a circulatory device system designed to provide information corresponding to cardiac parameters of a percutaneous circulatory support device and/or a patient.

### BACKGROUND

Percutaneous circulatory support devices such as blood pumps can provide transient cardiac support in patients whose heart function or cardiac output is compromised. Such devices may be delivered percutaneously from the femoral artery, retrograde through the descending aorta, over the aortic arch, through the ascending aorta across the aortic valve, and into the left ventricle. To assist a clinician using a circulatory support device during a medical procedure, a circulatory support device system may include one or more displays designed to provide easy-to-understand operational and alert information relating to one or more cardiac parameters of the percutaneous circulatory support device, the circulatory support device system and/or the patient. Accordingly, there is an ongoing need to provide circulatory support device systems including one or more visual displays designed to provide simplified visual information corresponding to cardiac parameters of a percutaneous circulatory support device and/or a patient. Circulatory support device systems including one or more visual displays designed to provide information corresponding to cardiac parameters of a percutaneous circulatory support device and/or a patient are disclosed herein.

### SUMMARY

This disclosure provides design, material, manufacturing method, and use alternatives for medical devices and/or systems. An example cardiac pump system includes a cardiac pump coupled to a console. Further, the console includes a first display including a first visual representation of a first parameter of the cardiac pump system. Further, the first display includes a first status indicator and a second status indicator corresponding to the first parameter. Further, the first display is configured to display the first status indicator when the first parameter satisfies a first condition, and wherein the first display is configured to display the second status indicator when the first parameter satisfies a second condition.

Alternatively or additionally to any of the embodiments above, wherein the console includes a second display configured to display information corresponding to the first parameter.

Alternatively or additionally to any of the embodiments above, wherein the first display includes a third status indicator corresponding to the first parameter, and wherein the first display is configured to display the third status indicator when the first parameter satisfies a third condition.

Alternatively or additionally to any of the embodiments above, wherein the first display includes a second visual representation of a second parameter of the cardiac pump system.

Alternatively or additionally to any of the embodiments above, wherein the first display includes a fourth status indicator, a fifth status indicator and a sixth status indicator corresponding to the second parameter.

Alternatively or additionally to any of the embodiments above, wherein the first display is configured to display only the first status indicator when the first parameter satisfies the first condition, and wherein the first display is configured to display only the second status indicator when the first parameter satisfies the second condition, and wherein the first display is configured to display only the third status indicator when the first parameter satisfies the third condition.

Alternatively or additionally to any of the embodiments above, wherein the first status indicator is represented by a first color, wherein the second status indicator is represented by a second color different from the first color, and wherein the third status indicator is represented by a third color different from the first color and the second color.

Alternatively or additionally to any of the embodiments above, wherein cardiac pump system further includes a processer configured to receive a data signal from a first sensor corresponding to the first parameter and updates the first status indicator, the second status indicator or both the first status indicator and the second status indicator based on the data signal.

Alternatively or additionally to any of the embodiments above, wherein the first display is configured to continue to display the first visual representation, the first status indicator and the second status indicator if the second display fails to display any information.

Alternatively or additionally to any of the embodiments above, wherein the first visual representation includes an icon.

Alternatively or additionally to any of the embodiments above, wherein the first visual representation includes a word.

Alternatively or additionally to any of the embodiments above, wherein the first visual representation corresponds to one or more of an electrical connection to the console, an optical connection to a pressure sensor, a power current to a pump, a speed of the pump, an arterial pressure, a power current to a motor, a speed of the motor, a rotations per minute measurement of the motor.

Alternatively or additionally to any of the embodiments above, wherein the first status indicator and the fourth status indicator are aligned along a first horizonal axis of the first display, the second status indicator and the fifth status indicator are aligned along a second horizonal axis of the first display and the third status indicator and the sixth status indicator are aligned along a third horizontal axis of the first display.

Alternatively or additionally to any of the embodiments above, wherein the first status indicator, the second status indicator, the third status indicator and the first visual representation are aligned along a first vertical axis of the first display.

Alternatively or additionally to any of the embodiments above, wherein the first visual representation is positioned between the second status indicator and the third status indicator.

Alternatively or additionally to any of the embodiments above, wherein the fourth status indicator, the fifth status indicator, the sixth status indicator and the second visual representation are aligned along a second vertical axis of the first display.

Alternatively or additionally to any of the embodiments above, wherein the second visual representation is positioned between the fifth status indicator and the sixth status indicator.

Alternatively or additionally to any of the embodiments above, wherein the first visual representation and the second visual representation are aligned along a fourth horizontal axis of the first display.

An example circulatory assist device system console includes a display including a visual parameter array configured to display information relating to a parameter of a circulatory assist device, wherein the visual parameter array includes a visual parameter representation corresponding to the parameter and a status indicator corresponding to a condition of the parameter, wherein the visual parameter representation and the status indicator are aligned along a vertical axis of the display and wherein a change in the condition of the parameter causes a movement of the status indicator along the vertical axis of the display.

Another example cardiac pump system console includes a first display including a plurality of visual parameter arrays configured to display information relating to a plurality of parameters of a circulatory assist device, wherein each of the visual parameter arrays includes a visual parameter representation corresponding to the parameter, a first status indicator corresponding to a first condition of the parameter and a second status indicator corresponding to a second condition of the parameter. The console also includes a second display. Further, the visual parameter arrays are aligned horizontally along the display. Further, the visual parameter representation, the first status indicator and the second status indicator of each array are aligned along a vertical axis of their respective visual parameter array.

Another example cardiac pump system includes a cardiac pump coupled to a console. The console includes a processor, a first display communicatively coupled to the processor and a second display communicatively coupled to the processor. Further, the second display includes a first visual representation of a first parameter of the cardiac pump system and a second visual representation of a second parameter of the cardiac pump system. Further, the second display includes a first status indicator, a second status indicator and a third status indicator corresponding to the first parameter. Further, the second display is configured to display the first status indicator when the first parameter satisfies a first condition, wherein the second display is configured to display the second status indicator when the first parameter satisfies a second condition, and wherein the second display is configured to display the third status indicator when the first parameter satisfies a third condition.

Another cardiac pump system includes a first display including a plurality of visual parameter arrays configured to display information relating to a plurality of parameters of a circulatory assist device, wherein each of the visual parameter arrays includes a visual parameter representation corresponding to the parameter, a first status indicator corresponding to a first condition of the parameter and a second status indicator corresponding to a second condition of the parameter. The system also includes a second display configured to display information corresponding to the first parameter, wherein the visual parameter representation, the first status indicator and the second status indicator of each array are configured to be displayed on the first display when no information is displayed on the second display.

Another cardiac pump system includes a cardiac pump coupled to a console. The console includes a processor, a first display communicatively coupled to the processor; and a second display communicatively coupled to the processor. Further, the first display includes a first visual representation corresponding to a parameter of the cardiac pump, wherein the first display includes a first status indicator representing a normal operating condition of the parameter, a second status indicator representing a warning condition of the parameter, and a third status indicator representing an alert condition of the parameter. Further, the status indicators are configured to be displayed in sequence from the first status indicator to the second status indicator to the third status indicator.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

FIG. 1 depicts a percutaneous circulatory support system, including a circulatory support device and its relative position in a heart of a patient;

FIG. 2 is a schematic block diagram of a console management system;

FIG. 3 is a schematic depiction of an example display of a circulatory support system;

FIG. 4 is a schematic depiction of an example display of a circulatory support system;

FIG. 5 is a schematic depiction of an example display of a circulatory support system;

FIG. 6 is a schematic depiction of an example display of a circulatory support system;

FIG. 7 is a schematic depiction of an example display of a circulatory support system;

FIGS. 8-10 illustrate multiple schematic views of an example display of a circulatory support system;

FIG. 11 is a schematic depiction of an example display of a circulatory support system;

FIG. 12 is a schematic depiction of an example display of a circulatory support system.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

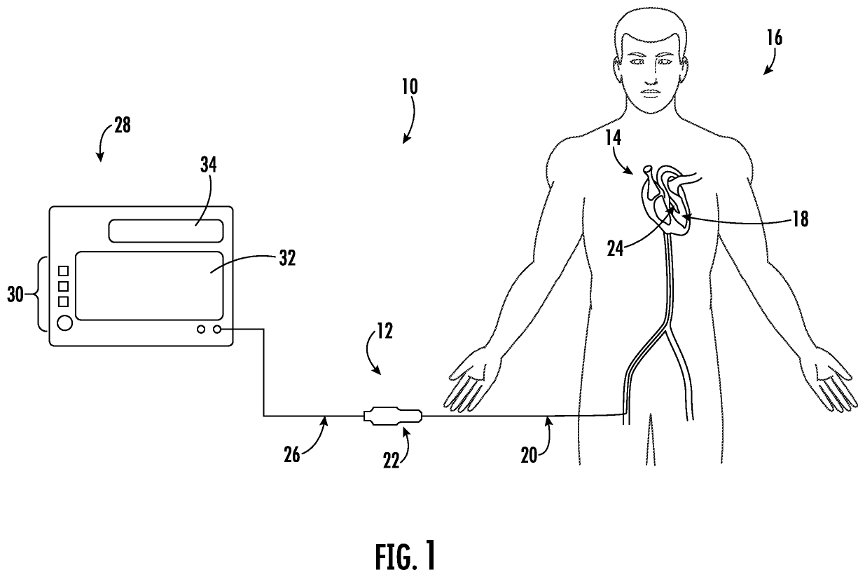

FIG. 1 illustrates an example percutaneous circulatory system 10 including a circulatory support device 12 positioned in the heart 14 of a patient 16. The circulatory support device 12 may include a flexible elongated catheter shaft 20 having a first end attached to a handle 22 and a second end attached to a blood pump 24. FIG. 1 illustrates the blood pump 24 positioned in the left ventricle 18 of the patient 16. The blood pump 24 may be delivered (e.g., tracked) to the ventricle 18 percutaneously over a guidewire. For example, the catheter shaft 20 and blood pump 24 may be tracked over a guidewire through the femoral artery and the descending aorta, over the aortic arch, through the ascending aorta, past the aortic valve and into the left ventricle 18.

In some examples, the shaft 20 of the circulatory support device 12 may include one or more blood inlets located on a distal region of the catheter shaft 20, and one or more blood outlets which are along a housing of the blood pump 24. In some examples, the blood pump 24 may be positioned within the heart 14 such that the one or more blood inlets positioned along the distal region of the catheter shaft 20 may be positioned in the left ventricle 18 and the one or more blood outlets located along the housing of the blood pump 24 may be positioned in the aorta. Additionally, the blood pump 24 may include an electrically powered motor that drives rotation of an impeller (positioned within the housing of the blood pump 24). In some examples, the motor may power the rotation of the impeller via electromagnetic induction. The spinning impeller may draw blood from the left ventricle (via the one or more blood inlets located on a distal region of the catheter shaft 2) into the aorta (via the one or more outflow inlets located along the housing of the blood pump 24). In other words, an electrically powered motor drives the impeller to pump blood from the left ventricle through the aortic valve and into the ascending aorta.

FIG. 1 further illustrates that a first end of the catheter shaft 20 may be attached to a handle 22. The handle 22 may include a distal end region attached to the catheter shaft 20 and a proximal end region attached to an electrical power cable 26. The electrical power cable 26 may include a distal end region connected to a console 28. It can be appreciated that the handle 22 may include one or more actuators (e.g., buttons, levers, dials, switches, etc.) designed to permit a clinician to control various functions of the blood pump 24. For example, a clinician may be able to control the speed of the motor and/or the impeller located in the blood pump 24 via actuation of one or more actuators located on the handle 22.

Additionally, the percutaneous circulatory system 10 may include one or more sensors positioned along the console 28, the handle 22, the catheter shaft 20 and/or the blood pump 24. The one or more sensors positioned within the console 28, the handle 22, along the catheter shaft 20 and/or the blood pump 24 may be designed to monitor blood pressures (e.g., arterial pressure, venous pressure), blood velocity, blood flowrate, or other parameters. Additionally, the one or more sensors positioned along the console 28, within the handle 22, the catheter shaft 20 and/or the blood pump 24 may be designed to monitor other parameters of the circulatory system 10, the circulatory support device 12 and/or the patient 16. Example parameters that may be sensed and monitored may include the electrical connection of the electrical cable 26 to the console 28, an optical and/or electrical connection to a pressure sensor positioned in the catheter shaft 20 and/or the blood pump 24, a power current to the blood pump 24, the speed of the impeller of the blood pump 24, an arterial pressure, a venous pressure, a power current to the motor, the speed of the motor, rotations per minute measurement of a motor, the voltage driving the motor, the back-EMF from the motor, etc. and any combination or temporal pattern of signals corresponding to one or more of the parameters listed herein. Further, additional parameters (e.g., flow through the blood pump 24) may be derived by processing combinations of any signal listed herein in a time dependent manner.

Additionally, FIG. 1 illustrates that the console 28 may include one or more control knobs (e.g., buttons, knobs, dials, etc.) 30 and/or one or more displays. For example, FIG. 1 illustrates the console 28 may include a first display 32 and a second display 34. It can be appreciated that the console 28 may include more than two displays. Additionally, while FIG. 1 illustrates the first display 32 and the second display 34 integrated into the console 28, it is contemplated that the circulatory system 10 may be designed such that the first display 32, the second display 34 or both the first display 32 and the second display 34 are separate, distinct components of the circulatory system 10. In other words, the first display 32, the second display 34 or both the first display 32 and the second display 34 may be separate stand-alone displays, apart from the console 28. In some examples, the first display 32 and the second display 34 may get their respective data from separate sources.

In some examples, the second display 34 may be designed to attach to the console 28 and/or the first display 32. For example, the first display 32 may be integrated into the console 28 while the second display 34 may be configured to attach to portion of the console 28. In yet other examples, both the first display 32 and the second display 34 may be a separate stand-alone display whereby the second display 34 may be configured to attach to the first display 32, or wherein the first display 32 may be configured to attach to the second display 34.

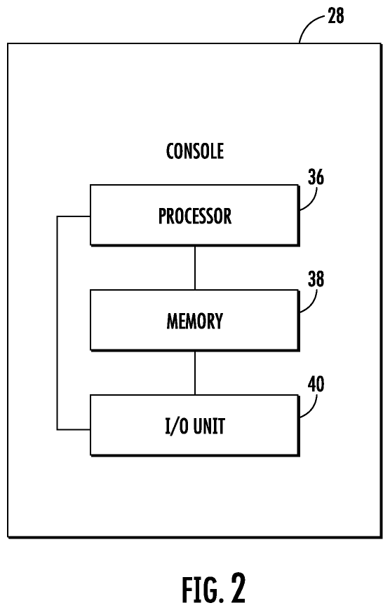

FIG. 2 illustrates that the console 28 may include, among other suitable components, one or more processors 36, memory 38, and an I/O unit 40. The processor 36 of the console 28 may include a single processor or more than one processor (e.g., a first processor 36 providing data/instructions to the first display 32 and a second processor 36 providing data instructions to a second display 34) working individually or with one another. The processor 36 may be configured to execute instructions, including instructions that may be loaded into the memory 38 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 36 of the console may be configured to execute program instructions. Program instructions may include, for example, firmware, microcode or application code that is executed by the processor 36, a microprocessor and/or microcontroller. The one or more processors 36 may be configured to each manage different functions. They may also be configured to concurrently perform the same functions (e.g., redundant system). Further yet, they may be configured such that a first processor 36 performs a given function and second processor 36 checks the result of the function of the first processor 36 for correctness (e.g., command-monitor system).

In some examples, the first display 32 may controlled primarily by the console's firmware control instructions and, therefore, may require relatively little processing power, relatively few instructions and very simple communication between the processor 36 and the display 32, compared to the second display 34 (e.g., a touch screen display 34), which may be controlled primarily by an embedded computer with a flexible and relatively complex communication protocol.

The memory 38 of the console 28 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory 38 may be or may include a non-transitory computer readable medium.

The I/O units 40 of the console 28 may include a single I/O component or more than one I/O component each working individually or with one another. Example I/O units 40 may be any type of communication port configured to communicate with other components of the circulatory system 10. Example types of I/O units 45 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

Additionally, the first display 32 and/or the second display 34 may include a CRT, LED, 3D display, other type of display, for example. The first display 32 and/or the second display 34 may present information relevant to functional and operational parameters of the circulatory system 10, circulatory support device 12 and/or physiological parameters of the patient 16 in a simple format useful to clinicians. For example, the first display 32 and/or the second display 34 may be configured to display graphical information relevant to the electrical connection of the electrical cable 26 to the console 28, an optical connection to a pressure sensor positioned in the catheter shaft 20 and/or the blood pump 24, a power current to the blood pump 24, the speed of the impeller of the blood pump 24, an arterial pressure, a venous pressure, a power current to the motor, the speed of the motor, rotations per minute measurement of a motor, the voltage driving the motor, the back-EMF from the motor, etc. and any combination or temporal pattern of signals corresponding to one or more of the parameters listed herein. Further, additional parameters (e.g., flow through the blood pump 24) may be derived by processing any combination or temporal pattern of signals corresponding to one or more of the parameters listed herein in a time dependent manner. Further, the first display 32 may indicate if the system 10 is operating normally, if the system 10 has detected a specific issue that may require additional (e.g., non-routine) procedures/adjustments, and/or if the system 10 has detected s specific issue that requires emergency procedures. Furthermore, the first display 32 may include information which conveys that a particular response (e.g., action) is needed. For example, the display 32 may be able to convey information that the blood pump 24 has failed and needs to be replaced immediately during a medical procedure.

In some examples, the circulatory system 10 may be designed such that the type and/or arrangement of graphical information displayed on the first display 32 may be different than the information displayed on the second display 34. For example, in some instances, the second display 34 may be designed to display detailed information about one or more functional, operational and/or physiological parameters relating to the circulatory system 10, circulatory support device 12 and/or the patient 16 while the first display 32 may be designed to provide a simplified, “global” information status summary for one or more functional, operational or physiological parameters relating to the circulatory system 10, circulatory support device 12 and/or the patient 16.

As can be appreciated from FIG. 1, in some examples, the first display 32 may assume a smaller-sized footprint within the console 28 compared to the relatively larger-sized second display 34. Accordingly, the first display 32 may be designed to provide a simplified, visual information summary designed to easily convey the status of one or more functional, operational and/or physiological parameters of the circulatory system 10, circulatory support device 12 and/or the patient 16. In some examples, the visual information displayed on the first display 32 may correspond, relate or correlate to one or more functional, operational and/or physiological parameters displayed on the second display 34. However, in other examples, the parameters displayed on the first display 32 may not relate to the to one or more parameters displayed on the second display 34.

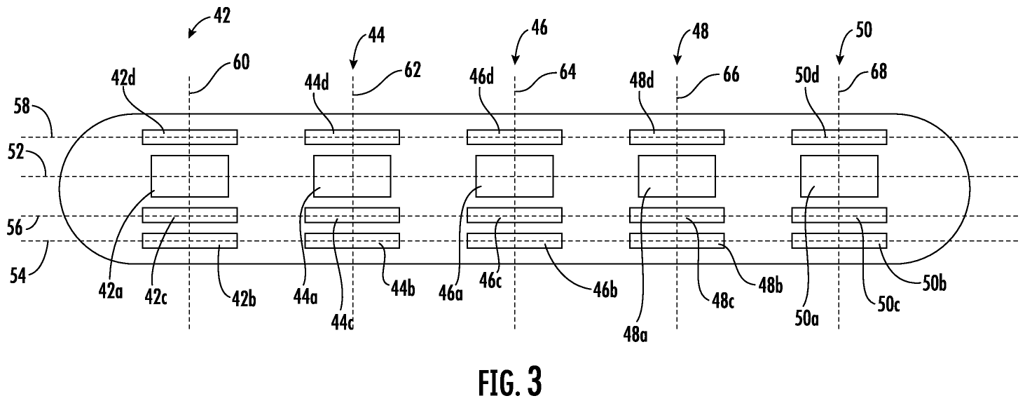

FIG. 3 is a schematic depiction of an example display of the first display 32 discussed herein. FIG. 3 illustrates an example arrangement and the relative position of one or more symbols (e.g., icons, visual representations, words, signs, visual alerts, etc.) which may be displayed (e.g., illuminated) on the display 32. As will be discussed in greater detail below, one or more of the symbols may be illuminated on the display 32 in a variety of different configurations.

The example display 32 illustrated in FIG. 3 may include one or more “visual parameter arrays” arranged side-by-side, relative to one another. For example, FIG. 3 illustrates that the display 32 may include a first visual parameter array 42, a second visual parameter array 44, a third visual parameter array 46, a fourth visual parameter array 48, and a fifth visual parameter array 50. It can be appreciated that each individual “visual parameter array” 42, 44, 46, 48, 50 may include a collection or cluster of symbols (e.g., icons, visual representations, words, signs, visual alerts, etc.) which together represent a particular functional or physiological parameter of the circulatory system 10 and/or the patient 16, in addition to providing information regarding the “status” of that particular parameter.

Additionally, each visual parameter array may include a “visual parameter representation” and one or more “parameter status indicators.” For example, the visual parameter array 42 illustrated in FIG. 3 may include a “visual parameter representation” 42a which may include a symbol (e.g., icons, visual representations, words, signs, visual alerts, etc.) relating to the motor of the blood pump 24 of the circulatory device 12, for example. Further, the visual parameter array 42 illustrated in FIG. 3 may also include one or more “parameter status indicators” 42b, 42c, 42d, each of which may include a symbol (e.g., icons, visual representations, words, signs, visual alerts, etc.) which may convey information relating to the operational state or condition of the motor of the blood pump 24, for example. It can be appreciated that a variety of various functional and physiological parameters of the circulatory system 10 and/or the patient 16 may be represented by the visual parameter arrays 42, 44, 46, 48, 50 shown in FIG. 3.

It can be further appreciated from FIG. 3 that the visual parameter representation and each parameter status indicator for a respective visual parameter array 42, 44, 46, 48, 50 may be arranged as a vertical “stack” on the display 32. For example, FIG. 3 illustrates that the visual parameter array 42 may include a first visual parameter representation 42a, a first parameter status indicator 42b, a second parameter status indicator 42c and a third status parameter indicator 42d, each of which is vertically stacked relative to one another along a vertical axis 60 of the display 32. Further, FIG. 3 illustrates that visual parameter array 44 may include a first visual parameter representation 44a, a first parameter status indicator 44b, a second parameter status indicator 44c and a third status parameter indicator 44d, each of which is vertically stacked relative to one another along a vertical axis 62 of the display 32. Further yet, FIG. 3 illustrates that visual parameter array 46 may include a first visual parameter representation 46a, a first parameter status indicator 46b, a second parameter status indicator 46c and a third status parameter indicator 46d, each of which is vertically stacked relative to one another along a vertical axis 64 of the display 32. Further, FIG. 3 illustrates that visual parameter array 48 may include a first visual parameter representation 48a, a first parameter status indicator 48b, a second parameter status indicator 48c and a third status parameter indicator 48d, each of which is vertically stacked relative to one another along a vertical axis 66 of the display 32. Further yet, FIG. 3 illustrates that visual parameter array 50 may include a first visual parameter representation 50a, a first parameter status indicator 50b, a second parameter status indicator 50c and a third status parameter indicator 50d, each of which is vertically stacked relative to one another along a vertical axis 68 of the display 32.

FIG. 3 illustrates that status indicators corresponding to each visual parameter array 42, 44, 46, 48, 50 may be represented by an elongated rectangular shape. For example, FIG. 3 illustrates each of first status indicators 42b, 44b, 46b, 48b, 50b, the second status indicators 42c, 44c, 46c, 48c, 50c and the third status indicators 42d, 44d, 46d, 48d, 50d represented as an elongated rectangular “bar.” Accordingly, as shown in FIG. 3, the first status indicators 42b, 44b, 46b, 48b, 50b may form a horizontal row of rectangular bars extending across the display 32 (each of which may be individually illuminated on the display 32), the second status indicators 42c, 44c, 46c, 48c, may form a horizontal row of rectangular bars extending across the display 32 (each of which may be individually illuminated on the display 32) and the third status indicators 42d, 44d, 46d, 48d, may form a horizontal row of rectangular bars extending across the display 32 (each of which may be individually illuminated on the display 32). However, it can be appreciated that any of the status indicators described herein may be represented by any shape on the display 32. For example, any of the status indicators (or visual parameter representations) may be represented by a square, triangle, rectangle, oval, circle, star, polygon, or any other shape.

Additionally, it can be appreciated that any of the symbols (e.g., any symbol utilized as a visual parameter representation or any status indicator discussed herein) may be represented using a color. For example, in some instances, the first status indicators 42b, 44b, 46b, 48b, 50b may be illuminated using the color white, the second status indicators 42c, 44c, 46c, 48c, 50c may be illuminated using the color yellow, and the third status indicators 42d, 44d, 46d, 48d, 50d may be illuminated using the color red. It can be appreciated that any color or arrangement of colors may be utilized to represent and/or illuminate any symbol (e.g., any symbol utilized as a visual parameter representation or any status indicator) on the display 32.

Additionally, the example display 32 may be configured such that any individual symbol (e.g., icon, visual representation, word, sign, visual alert, etc.) may be displayed individually or in combination with any other symbol on the display 32. Further, the symbols (e.g., icons, visual representations, words, signs, visual alerts, etc.) displayed on the display 32 may relate (e.g., correspond, correlate, etc.) to any information displayed on the first display 32.

While FIG. 3 illustrates the display 32 including five visual parameter arrays 42, 44, 46, 48, 50, it can be appreciated that the display 32 may include fewer than five visual parameter arrays (each of which may include one or more visual parameter representations and/or status indicators). Alternatively, it can be appreciated that the display 32 may include more than five visual parameter arrays (each of which may include one or more visual parameter representations and/or status indicators). For example, the display 32 may include 1, 2, 3, 4, 5, 6, 7, 8, 9, 10 or more visual parameter arrays (each of which may include one or more visual parameter representations and/or status indicators).

FIG. 3 further illustrates that, in some examples, the one or more visual parameter representations, first parameter status indicators, the second parameter status indicators and/or the third status parameter indicators discussed in FIG. 3 may be horizontally aligned with one another, respectively. For example, FIG. 3 illustrates that, in some examples, the visual parameter representations 42a, 44a, 46a, 48a, 50a may be aligned horizontally relative to one another along a horizontal axis 52 of the display 32. Further, FIG. 3 illustrates that, in some examples, the first status indicators 42b, 44b, 46b, 48b, 50b may be aligned horizontally relative to one another along a horizontal axis 54 of the display 32. Further, FIG. 3 illustrates that, in some examples, the second status indicators 42c, 44c, 46c, 48c, 50c may be aligned horizontally relative to one another along a horizontal axis 56 of the display 32. Further, FIG. 3 illustrates that, in some examples, the third status indicators 42d, 44d, 46d, 48d, 50d may be aligned horizontally relative to one another along a horizontal axis 58 of the display 32.

FIG. 3 illustrates that, in some examples, the visual parameter representation and the status indicators may be arranged in a particular order along the vertical axis of that respective visual parameter array. For example, FIG. 3 illustrates that the visual parameter representation 42a may be positioned between the second status indicator 42c and the third status indicator 42d of the first visual parameter array 42. FIG. 3 further illustrates that each of the other visual parameter arrays 44, 46, 48, 50 shown in FIG. 3 may have a similar arrangement as the visual parameter array 42, whereby the respective visual parameter representations 44a, 46a, 48a, 50a are positioned between the second status indicator 44c, 46c, 48c, 50c and the third status indicator 44d, 46d, 48d, 50d of that respective visual status array 44, 46, 48 50.

It can be appreciated that the visual parameter representations and the status indicators for any individual visual parameter array 42, 44, 46, 48, 50 discussed herein may be arranged in any given configuration. For example, for any visual parameter array 42, 42, 46, 48, 50, the visual parameter representations 42a, 44a, 46a, 48a, 50a may be positioned in any order relative to any of the status indicators to which it corresponds. For example, the visual parameter representation 42a may be positioned in any order among the first status indicator 42b, second status indicator 42c and third status indicator 42d of the first visual parameter array 42. For example, the visual parameter representation 42a, first status indicator 42b, second status indicator 42c and third status indicator 42d may be ordered (from bottom to top, along the vertical axis 60) as: visual parameter representation 42a to first status indicator 42b to second status indicator 42c to third status indicator 42d; or may be ordered as first status indicator 42b to visual parameter representation 42a to second status indicator 42c to third status indicator 42d; or may be ordered as first status indicator 42b to second status indicator 42c to third status indicator 42d to visual parameter representation 42a. It can be appreciated that the vertical arrangement of the visual parameter representations and the status indicators of the other individual visual parameter arrays 44, 46, 48, 50 may be similar to that of the vertical arrangement of the visual parameter 42a discussed herein.

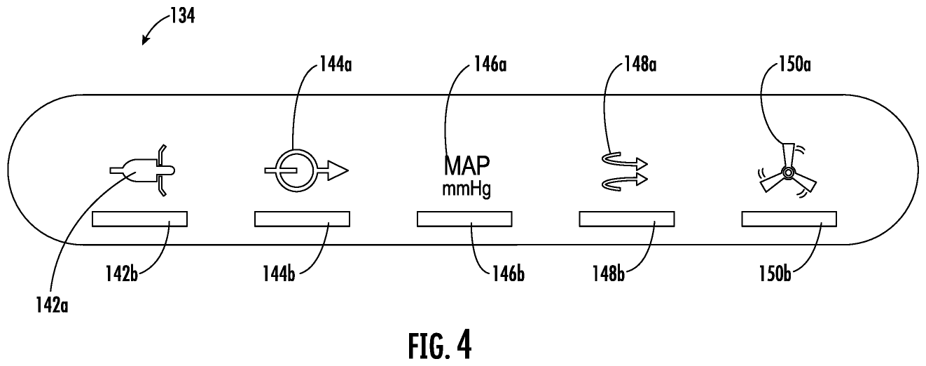

FIG. 4 illustrates an example display 134 which may be similar in form and function to the display 32 discussed herein. FIG. 4 illustrates visual parameter representations 142a, 144a, 146a, 148a, 150a which are horizontally aligned with one another along the display 134 (similar to the horizontal alignment of the visual parameter representations 42a, 44a, 46a, 48a, 50a discussed with respect to FIG. 3). FIG. 4 further illustrates that each of the visual parameter representations 142a, 144a, 146a, 148a, 150a may include a symbol (e.g., icon) which may represent a particular functional or operational parameter of the circulatory system 10 or a physiological parameter of the patient 16. For example, the visual parameter representation 142a may include a symbol which represents the electrical connection of the electrical cable 26 to the console 28, the visual parameter representation 144a may include a symbol which represents the position of the blood pump 24 within the patient 16, the visual parameter representation 148a may include a symbol which represents the amount of suction being applied by the blood pump 24 within the heart 14 of the patient 16, and the visual parameter representation 150a may include a symbol which represents the speed of the motor driving the impeller of the blood pump 24. Further, FIG. 4 illustrates that, in some examples, the visual parameter representations 142a, 144a, 146a, 148a, 150a may include a word which describes a particular functional or operational parameter of the circulatory system 10 or a physiological parameter of the patient 16. For example, the visual parameter representation 146a shown in FIG. 4 includes the word “MAP”, which represents the “Mean Arterial Pressure” within the aorta of the patient's heart 16. However, it is contemplated that the visual parameter representation 146a may be represented by an icon, similar to the other visual parameter representations 142a, 144a, 148a, 150a shown in FIG. 4.

FIG. 4 further illustrates the first status indicators 142b, 144b, 146b, 148b, 150b may be vertically aligned with each of the visual parameter representations 142a, 144a, 146a, 148a, 150a. The first status indicators 142b, 144b, 146b, 148b, 150b may be similar to the first status indicators 42b, 44b, 46b, 48b, 50b described with respect to FIG. 3. FIG. 4 further illustrates that first status indicators 142b, 144b, 146b, 148b, 150b may be horizontally aligned with one another, similar to the first status indicators 42b, 44b, 46b, 48b, 50b described with respect to FIG. 3.

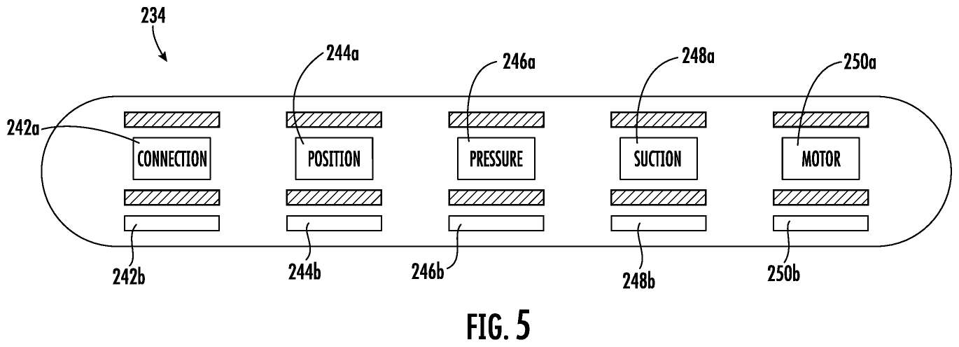

FIG. 5 illustrates another example display 234 which may be similar in form and function to the displays 34, 134 discussed herein. FIG. 5 illustrates visual parameter representations 242a, 244a, 246a, 248a, 250a which are horizontally aligned with one another along the display 234 (similar to the horizontal alignment of the visual parameter representations 42a, 44a, 46a, 48a, 50a discussed with respect to FIG. 3). FIG. 5 further illustrates that each of the visual parameter representations 242a, 244a, 248a, 250a may include a word which represents (e.g., describes, indicates) a particular functional or operational parameter of the circulatory system 10 or a physiological parameter of the patient 16. For example, the visual parameter representation 242a may include the word “CONNECTION” which may represent the electrical connection of the electrical cable 26 to the console 28, the visual parameter representation 244a may include the word “POSITION” which may represent the position of the blood pump 24 within the patient 16, the visual parameter representation 246a may include the word “PRESSURE”, which may represent a particular blood pressure (e.g., mean arterial pressure, etc.) within the patient's heart 16, the visual parameter representation 248a may include the word “SUCTION” which may represent the amount of suction being applied by the blood pump 24 within the heart 14 of the patient 16, and the visual parameter representation 250a may include the word “MOTOR” which represents a the speed of the motor driving the impeller of the blood pump 24.

FIG. 5 further illustrates the first status indicators 242b, 244b, 246b, 248b, 250b may be vertically aligned with each of the visual parameter representations 242a, 244a, 246a, 248a, 250a. The first status indicators 242b, 244b, 246b, 248b, 250b may be similar to the first status indicators 42b, 44b, 46b, 48b, 50b described with respect to FIG. 3. FIG. 4 further illustrates that the first status indicators 242b, 244b, 246b, 248b, 250b may be horizontally aligned with one another, similar to the first status indicators 42b, 44b, 46b, 48b, 50b described with respect to FIG. 3.

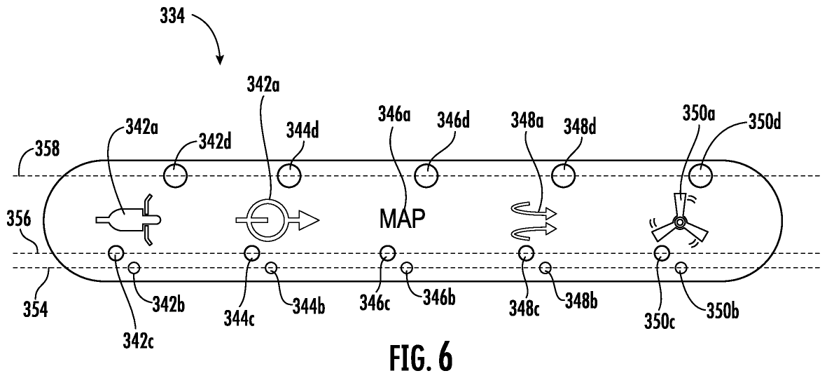

FIG. 6 illustrates another example display 334 which may be similar in form and function to other displays discussed herein. FIG. 6 illustrates visual parameter representations 342a, 344a, 346a, 348a, 350a which are horizontally aligned with one another along the display 334 (similar to the horizontal alignment of the visual parameter representations 42a, 44a, 46a, 48a, 50a discussed with respect to FIG. 3). FIG. 6 further illustrates that each of the visual parameter representations 342a, 344a, 348a, 350a may include a symbol (e.g., icon) which may represent a particular functional or operational parameter of the circulatory system 10 or a physiological parameter of the patient 16 (similar to the icons discussed with respect to FIG. 4). Further, FIG. 6 further illustrates the first status indicators 342b, 344b, 346b, 348b, 350b represented by a circle, whereby each of the circle-shaped status indicators 342b, 344b, 346b, 348b, 350b are aligned with one another along the horizontal axis 354. Further, FIG. 6 illustrates second status indicators 342c, 344c, 346c, 348c, 350c represented by a larger circle (relative to the circle representing the status indicators 342b, 344b, 346b, 348b, 350b), whereby each of the circle-shaped status indicators 342c, 344c, 346c, 348c, 350c are aligned with one another along the horizontal axis 356. Further, FIG. 6 illustrates third status indicators 342d, 344d, 346d, 348d, 350d represented by an even larger circle (relative to the circle representing the status indicators 342b, 344b, 346b, 348b, 350b and status indicators 342c, 344c, 346c, 348c, 350c), whereby each of the circle-shaped status indicators 342d, 344d, 346d, 348d, 350d are aligned with one another along the horizontal axis 358. Additionally, FIG. 6 illustrates that the first status indicators 342b, 344b, 346b, 348b, 350b, the second status indicators 342c, 344c, 346c, 348c, 350c and the third status indicators 342d, 344d, 346d, 348d, 350d shown on display 334 are not aligned with one another, respectively, along a given vertical axis. As described herein, any of the status indicators (or visual parameter representations) illustrated in FIG. 6 may be represented by any shape, including a square, triangle, rectangle, oval, circle, star, polygon, etc.

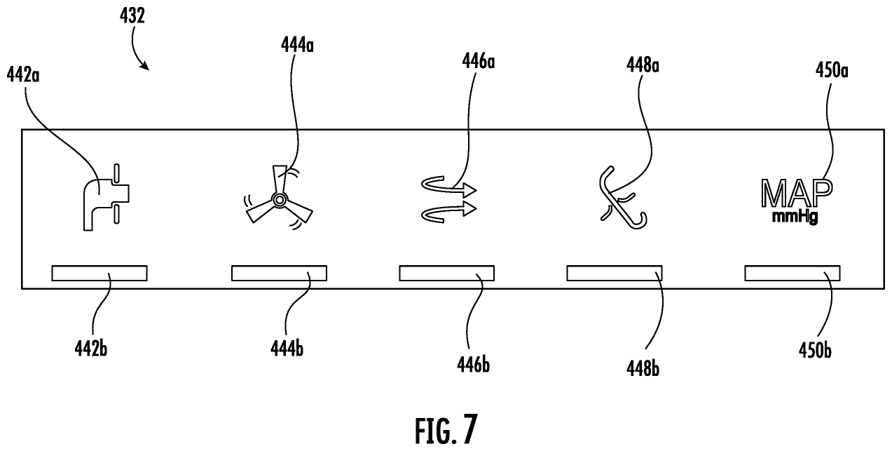

FIG. 7 illustrates an example display 432 which may be similar in form and function to the display 32 discussed herein. FIG. 7 illustrates visual parameter representations 442a, 444a, 446a, 448a, 450a which are horizontally aligned with one another along the display 432 (similar to the horizontal alignment of the visual parameter representations 42a, 44a, 46a, 48a, 50a discussed with respect to FIG. 3). FIG. 7 further illustrates that each of the visual parameter representations 442a, 444a, 446a, 448a, 450a may include a symbol (e.g., icon) which may represent a particular functional or operational parameter of the circulatory system 10 or a physiological parameter of the patient 16. For example, the visual parameter representation 442a may include a symbol which represents the electrical connection of the electrical cable 26 to the console 28, the visual parameter representation 444a may include a symbol which represents the speed of the motor driving the impeller of the blood pump 24, the visual parameter representation 446a may include a symbol which represents the amount of suction being applied by the blood pump 24 within the heart 14 of the patient 16 and the visual parameter representation 448a may include a symbol which represents the position of the blood pump 24 within the patient 16. Further, FIG. 7 illustrates that, in some examples, the visual parameter representations 442a, 444a, 446a, 448a, 450a may include a word which describes a particular functional or operational parameter of the circulatory system 10 or a physiological parameter of the patient 16. For example, the visual parameter representation 450a shown in FIG. 7 includes the word “MAP”, which represents the “Mean Arterial Pressure” within the aorta of the patient's heart 16. However, it is contemplated that the visual parameter representation 450a may be represented by an icon, similar to the other visual parameter representations 442a, 444a, 446a, 448a shown in FIG. 7.

FIG. 7 further illustrates the first status indicators 442b, 444b, 446b, 448b, 450b may be vertically aligned with each of the visual parameter representations 442a, 444a, 446a, 448a, 450a. The first status indicators 442b, 444b, 446b, 448b, 450b may be similar to the first status indicators 42b, 44b, 46b, 48b, 50b described with respect to FIG. 3. FIG. 7 further illustrates that first status indicators 442b, 444b, 446b, 448b, 450b may be horizontally aligned with one another, similar to the first status indicators 42b, 44b, 46b, 48b, 50b described with respect to FIG. 3.

As discussed herein, each of the visual parameter arrays 42, 44, 46, 48, 50 illustrated and described herein, may identify (e.g., represent, symbolize, etc.) a particular functional or operational parameter of the circulatory system 10 or a physiological parameter the patient 16, while also conveying information relating to the “status” of that particular parameter during a medical procedure. For example, the display 32 may be configured present status information relating to a functional parameter of the circulatory system 10 or a physiological parameter the patient 16 in a simple, easy-to-understand format such that a clinician my easily determine from the display 32 the status of each parameter represented by the visual parameter arrays 42, 44, 46, 48, 50.

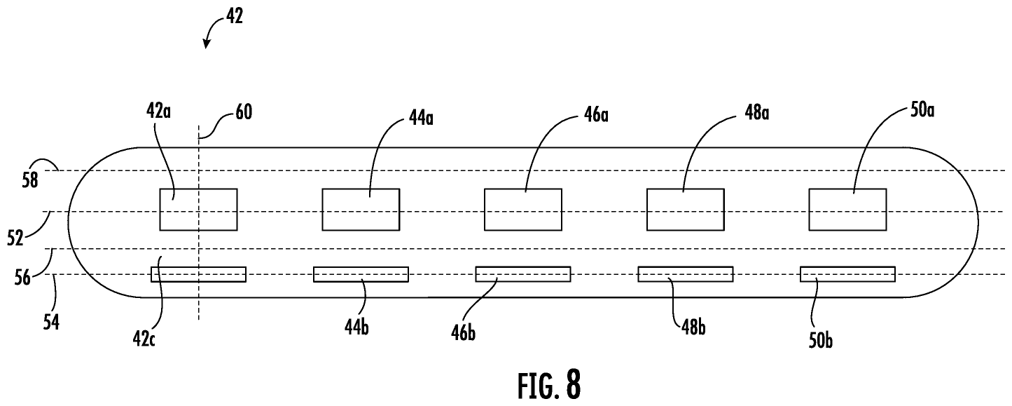

Accordingly, in some instances, the first, second and third status indicators of a given visual parameter array may provide real-time, stepwise (e.g., progressive), status information (e.g., an alert indication) corresponding to a functional or a physiological parameter of the system 10 or patient 16. FIGS. 8-10 illustrate the display 32 progressing through a stepwise status indication (e.g., an alert indication) corresponding to an example parameter (e.g., electrical connection between the electrical cable 26 and console 28) of the system 10. Accordingly, in some instances the visual parameter representation 42a shown in FIG. 8 may include a symbol (e.g., the icon 142a shown in FIG. 3) which conveys to a clinician that the visual parameter array 42 represents the electrical connection between the electrical cable 26 and console 28 of the system 10. Further, FIG. 8 illustrates the display 32 displaying the first status indicator 44b (and not displaying the status indicators 44c, 44d), positioned below the visual parameter representation 42a on the horizontal axis 54. The display of the first status indicator 44b may convey that the status of the electrical connection between the electrical cable 26 and console 28 is in an “informational/advisory” (e.g., normal, routine, etc.) operating condition (e.g., a sufficient amount of current is being transferred between the console 28 and the circulatory support device 12 along electrical cable 26). In other words, displaying any of the first status indicators 42b, 44b, 46b, 48b, 50b may convey to a clinician that the parameter to which the first status indicator 42b, 44b, 46b, 48b, 50b relates is operating in a normal (e.g., routine) operating condition.

Further, in some examples, the processing system of the system 10 may be programmed to define a range of values, minimum threshold, maximum threshold, etc. that defines the “informational/advisory” operating condition of the particular parameter to which the given first indicator 42b, 44b, 46b, 48b, 50b relates. As illustrated in FIG. 8, when each of the first status indicator 42b, 44b, 46b, 48b, 50b are displayed (e.g., a solid row of first indicators 42b, 44b, 46b, 48b, 50b are displayed along the horizontal axis 54), the display 32 is conveying to a clinician that all of the parameters represented by the visual parameter arrays 42, 44, 46, 48, 50 are operating in a normal operating condition and no user action is needed.

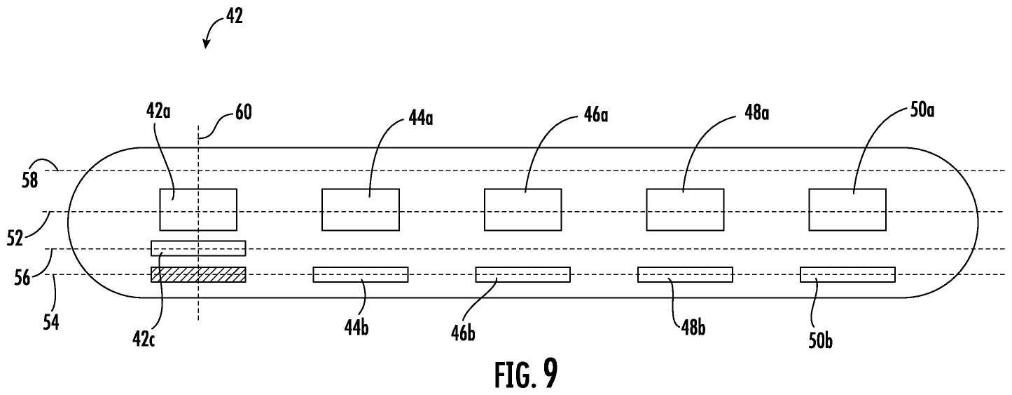

FIG. 9 illustrates the display 32 displaying the second status indicator 42c of the parameter represented by the visual parameter representation 42a (e.g., the electrical connection between the electrical cable 26 and console 28). FIG. 9 illustrates the status of the visual parameter representation 42a has changed from an informational/advisory operating condition (represented by the status indicator 42b) to a “serious” (e.g., early warning condition) (represented by the status indicator 42c) which may indicate that the functional, operational or physiological parameter represented by the visual parameter representation 42a has moved out of the informational/advisory operating range and into a serious condition, whereby user action maybe be required and the parameter may need improvement, adjustment, etc. (e.g., the electrical current being transferred between the console 28 and the circulatory support device 12 along electrical cable 26 may be in compromised condition).

Further, in some examples, the processing system of the system 10 may be programmed to define a range of values, minimum threshold, maximum threshold, etc. that defines the “serious” operating condition of the particular parameter to which the given second status indicators 42c, 44c, 46c, 48c, 50c relate.

FIG. 9 illustrates that the second status indicator 42c (positioned on the horizontal axis 56) is being displayed while the status indicator 42b (which was positioned on the horizontal axis 54) is no longer displayed. In other words, comparing the display shown in FIG. 8 to the display shown in FIG. 9 illustrates that the “status” of the parameter (e.g., the electrical connection between the electrical cable 26 and console 28) represented by the visual parameter representation 42a (e.g., the icon 142a shown in FIG. 3) has moved up the vertical axis 60 of the visual parameter array 42. As illustrated in FIG. 9, a clinician may be able to quickly look at the display 32 shown in FIG. 9 and understand that the status for the parameter represented by the visual parameter representation 42a has shifted from the first status indicator 42b to the second status indicator 42c. Further, it can be appreciated that the display 32 shown in FIG. 9 that the status of the parameters corresponding to the remaining visual parameter arrays 44, 46, 48, 50 remains in an informational/advisory operating condition.

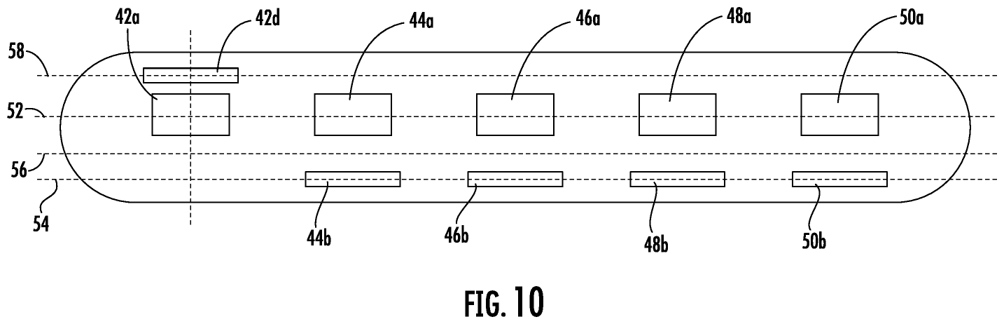

FIG. 10 illustrates the display 32 displaying the status indicator 42d of the parameter represented by the visual parameter representation 42a (e.g., the electrical connection between the electrical cable 26 and console 28). FIG. 10 illustrates the status of the visual parameter representation 42a has changed from a serious condition to a “critical” condition which may indicate that the functional, operational or physiological parameter represented by the visual parameter representation 42a has moved out of the early warning range and into a critical condition, whereby immediate user action is needed and the parameter needs to be changed and/or the medical procedure may need to be stopped (e.g., a condition in which the circulatory support device 12 is no longer receiving power from the console 28 and, therefore, the blood pump 24 cannot function).

FIG. 10 illustrates the third status indicator 42d (positioned on the horizontal axis 58) is being displayed while the status indicator 42c (which was positioned on the horizontal axis 56 in FIG. 9) is no longer displayed. In other words, comparing the display shown in FIG. 9 to the display shown in FIG. 10 illustrates that the “status” of the parameter (e.g., the electrical connection between the electrical cable 26 and console 28) represented by the visual parameter representation 42a (e.g., the icon 142a shown in FIG. 3) has moved up the vertical axis 60 of the visual parameter array 42. As illustrated in FIG. 10, a clinician may be able to quickly look at the display 32 shown in FIG. 10 and understand that the status for the parameter represented by the visual parameter representation 42a has shifted from the second status indicator 42c to the third status indicator 42d. Further, it can be appreciated from the display 32 shown in FIG. 10 that the status of the parameters corresponding to the remaining visual parameter arrays 44, 46, 48, 50 remain in an informational/advisory operating condition.

Additionally, in some examples, the non-display of a status indicator within a visual parameter arrays 42, 44, 46, 48, 50 may convey to the clinician that the status of the parameter represented by the visual parameter representation is unknown or has yet to be determined at that time.

Further, in some examples, the processing system of the system 10 may be programmed to define a range of values, minimum threshold, maximum threshold, etc. that defines the “critical” operating condition of the particular parameter to which the given second status indicators 42d, 44d, 46d, 48d, 50d relate.

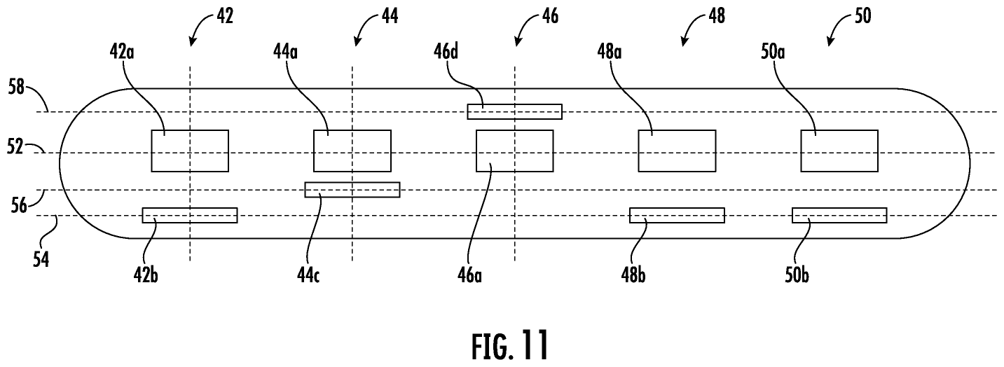

FIG. 11 illustrates another example whereby the display 32 conveys information regarding the condition of functional, operational or patient parameters by displaying different status indicators for multiple visual parameter arrays 42, 44, 46, 48, 50. FIG. 11 illustrates how the display 32 may be designed to present the status of system 10 and/or patient 16 parameters an easy-to-understand format such that a clinician my easily determine from the display 32 the status of each parameter represented by the visual parameter arrays 42, 44, 46, 48, 50.

For example, FIG. 11 illustrates the first status indicators 42b, 50b are displayed on the horizontal axis 54, whereby the display of the first status indicators 42b, 50b conveys to a clinician that the parameters represented by the visual parameter representations 42a, 50a are operating in an “informational/advisory” operating status. FIG. 11 further illustrates the second status indicator 44c is displayed on the horizontal axis 56, whereby the display of the second status indicator 44c conveys to a clinician that the parameter represented by the visual parameter representations 44a is operating in a “serious” operating status. Further yet, FIG. 11 illustrates the third status indicator 46d is displayed on the horizontal axis 58, whereby the display of the third status indicator 46d conveys to a clinician that the parameter represented by the visual parameter representation 46a is operating in a “critical” operating status. Further yet, FIG. 11 illustrates that no status indicator is displayed for the visual parameter array 48. Accordingly, because no status indicator is displayed for the visual parameter array 48, it may be understood that the status of the parameter represented by the visual parameter representation 48a is unknown. For example, if the visual parameter representation 48a represents the position of the blood pump within a patient, the non-display of a status indicator within the visual parameter array 48 may convey to a clinician that the position of the blood pump with the patient is unknown at that time. Further, if the position of the blood pump were to become known, a status indicator may appear within the visual parameter array 48 (as described herein), thereby conveying the operational status of the position of the blood pump within the patient to the clinician. It can be appreciated that the display configuration illustrated in FIG. 11 would allow a clinician to quickly assess that the parameters represented by the visual parameter representations 42a, 50a are operating in an informational/advisory condition, the parameter represented by the visual parameter representations 44a may need to be adjusted, the parameter represented by the visual parameter representation 46a may need to be changed immediately or that the medical procedure needs to be stopped and the status of the parameter represented by the visual parameter representation 48a is unknown.

In some examples, it can be appreciated that the status indicators within a given visual parameter array may remain displayed while other status indicators of the visual parameter array are displayed. In other words, the display 32 may be configured to operate such that the first status indicator may be displayed during a normal operating condition, the second status indicator may be displayed during a serious condition (whereby the first status indicator remains displayed along with the second status indicator) and the third status indicator may be displayed during a critical condition (whereby the first status indicator and the second status indicator may remain displayed along with the third indicator).

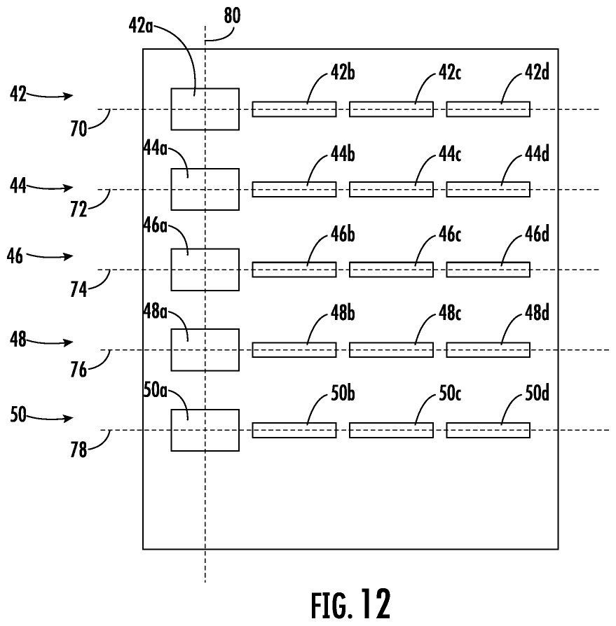

FIG. 12 illustrates an alternative arrangement of the visual parameter arrays 42, 44, 46, 48, 50 discussed with respect to FIG. 3. FIG. 12 illustrates that the display 32 discussed with respect to FIG. 3 may be arranged such that the visual parameter representations 42a, 44a, 46a, 48a, 50a are aligned vertically with respect to one another. FIG. 12 illustrates the visual parameter representations 42a, 44a, 46a, 48a, 50a aligned vertically along the vertical axis 80. Further, FIG. 12 illustrates that each of the first, second and third status indicators corresponding to each individual visual parameter representation 42a, 44a, 46a, 48a, 50a may be horizontally aligned with their respective visual parameter representation 42a, 44a, 46a, 48a, 50a on a horizontal axis. For example, FIG. 12 illustrates the visual parameter representation 42a aligned horizontally with the first status indicator 42b, the second horizontal indicator 42c and the third horizontal indicator 42d along the horizontal axis 70. FIG. 12 further illustrates the visual parameter representation 44a aligned horizontally with the first status indicator 44b, the second horizontal indicator 44c and the third horizontal indicator 44d along the horizontal axis 72. FIG. 12 further illustrates the visual parameter representation 46a aligned horizontally with the first status indicator 46b, the second horizontal indicator 46c and the third horizontal indicator 46d along the horizontal axis 74. FIG. 12 further illustrates the visual parameter representation 48a aligned horizontally with the first status indicator 48b, the second horizontal indicator 48c and the third horizontal indicator 48d along the horizontal axis 76. FIG. 12 further illustrates the visual parameter representation 50a aligned horizontally with the first status indicator 50b, the second horizontal indicator 50c and the third horizontal indicator 50d along the horizontal axis 78.

1. A cardiac pump system, comprising:
a cardiac pump coupled to a console, the console comprising:
a first display including a first visual representation of a first parameter of the cardiac pump system;
wherein the first display includes a first status indicator and a second status indicator corresponding to the first parameter;
wherein the first display is configured to display the first status indicator when the first parameter satisfies a first condition, and wherein the first display is configured to display the second status indicator when the first parameter satisfies a second condition.

2. The system of claim 1, wherein the console includes a second display configured to display information corresponding to the first parameter.

3. The system of claim 2, wherein the first display includes a third status indicator corresponding to the first parameter, and wherein the first display is configured to display the third status indicator when the first parameter satisfies a third condition.

4. The system of claim 3, wherein the first display includes a second visual representation of a second parameter of the cardiac pump system.

5. The system of claim 4, wherein the first display includes a fourth status indicator, a fifth status indicator and a sixth status indicator corresponding to the second parameter.

6. The system of claim 5, wherein the first display is configured to display only the first status indicator when the first parameter satisfies the first condition, and wherein the first display is configured to display only the second status indicator when the first parameter satisfies the second condition, and wherein the first display is configured to display only the third status indicator when the first parameter satisfies the third condition.

7. The system of claim 6, wherein the first status indicator is represented by a first color, wherein the second status indicator is represented by a second color different from the first color, and wherein the third status indicator is represented by a third color different from the first color and the second color.

8. The system of claim 1, wherein cardiac pump system further includes a processer configured to receive a data signal from a first sensor corresponding to the first parameter and updates the first status indicator, the second status indicator or both the first status indicator and the second status indicator based on the data signal.

9. The system of claim 2, wherein the first display is configured to continue to display the first visual representation, the first status indicator and the second status indicator if the second display fails to display any information.

10. The system of claim 1, wherein the first visual representation includes an icon.

11. The system of claim 1, wherein the first visual representation includes a word.

12. The system of claim 1, wherein the first visual representation corresponds to one or more of an electrical connection to the console, an optical connection to a pressure sensor, a power current to a pump, a speed of the pump, an arterial pressure, a power current to a motor, a speed of the motor, a rotations per minute measurement of the motor.

13. The system of claim 5, wherein the first status indicator and the fourth status indicator are aligned along a first horizonal axis of the first display, the second status indicator and the fifth status indicator are aligned along a second horizonal axis of the first display and the third status indicator and the sixth status indicator are aligned along a third horizontal axis of the first display.

14. The system of claim 13, wherein the first status indicator, the second status indicator, the third status indicator and the first visual representation are aligned along a first vertical axis of the first display.

15. The system of claim 14, wherein the first visual representation is positioned between the second status indicator and the third status indicator.

16. The system of claim 15, wherein the fourth status indicator, the fifth status indicator, the sixth status indicator and the second visual representation are aligned along a second vertical axis of the first display.

17. The system of claim 16, wherein the second visual representation is positioned between the fifth status indicator and the sixth status indicator.

18. The system of claim 17, wherein the first visual representation and the second visual representation are aligned along a fourth horizontal axis of the first display.

19. A circulatory assist device system console, comprising:
a display including a visual parameter array configured to display information relating to a parameter of a circulatory assist device;
wherein the visual parameter array includes a visual parameter representation corresponding to the parameter and a status indicator corresponding to a condition of the parameter;
wherein the visual parameter representation and the status indicator are aligned along a vertical axis of the display;
wherein a change in the condition of the parameter causes a movement of the status indicator along the vertical axis of the display.

20. A cardiac pump system console, comprising:
a first display including a plurality of visual parameter arrays configured to display information relating to a plurality of parameters of a circulatory assist device, wherein each of the visual parameter arrays includes a visual parameter representation corresponding to the parameter, a first status indicator corresponding to a first condition of the parameter and a second status indicator corresponding to a second condition of the parameter; and
a second display;
wherein the visual parameter arrays are aligned horizontally along the display;
wherein the visual parameter representation, the first status indicator and the second status indicator of each array are aligned along a vertical axis of their respective visual parameter array.
